(19) United States
(12) Patent Application Publication
(10) Pub. No.: US 2025/0054626 A1
(43) Pub. Date: Feb. 13, 2025

(54) AUTOMATED IDENTIFICATION OF THE DIAGNOSTIC CRITERIA IN NATURAL LANGUAGE DESCRIPTIONS OF PATIENT BEHAVIOR FOR COMBINING INTO A TRANSPARENT DIAGNOSTIC DECISION

(71) Applicant: Arizona Board of Regents on Behalf of the University of Arizona, Tucson, AZ (US)

(72) Inventor: Gondy Leroy, Tucson, AZ (US)

(21) Appl. No.: 18/797,060

(22) Filed: Aug. 7, 2024

Related U.S. Application Data
(60) Provisional application No. 63/518,063, filed on Aug. 7, 2023.

(51) Int. Cl. G16H 50/20; G16H 50/70

(52) U.S. Cl. CPC G16H 50/20; G16H 50/70

(57) ABSTRACT

A system that uses a rule-based parser and/or one or more machine learning models—e.g., a bidirectional gated recurrent unit (BiGRU) model, a hybrid bidirectional long short-term memory (BiLSTM-H) model, and/or a multilabel BiLSTM (BiLSTM-M) model, a large language model (LLM)—to label natural language sentences as indicative of diagnostic criteria used to diagnose and/or assess the severity of a mental disorder or other medical condition. In some embodiments, the system also determines whether the identified diagnostic criteria are indicative of a disorder under established medical guidelines and provides a final diagnostic label. By outputting both a diagnosis consistent with established medical guidelines and an understanding of the identified diagnostic criteria used to make that diagnosis, the disclosed system provides more clinical value to practitioners than the existing “black box” models used for medical diagnoses.

### CROSS-REFERENCE TO RELATED APPLICATIONS

This application claims priority to U.S. Prov. Pat. Appl. No. 63/518,063, filed Aug. 7, 2023, which is hereby incorporated by reference.

### FEDERAL FUNDING

This invention was made with government support under Grant No. MH124935 awarded by National Institutes of Health, and Grant No. HS024988 awarded by AHRQ. The government has certain rights in the invention.

### BACKGROUND

Machine learning is increasingly employed to diagnose a variety of mental disorders and other medical conditions. However, existing machine learning algorithms are usually trained to assign a single label using a black-box approach that does not provide a rationale for the label. Meanwhile, existing machine algorithms can be prone to finding spurious correlations and relying on irrelevant information. Together, the possibility of misdiagnosis and the lack of explainable results limits both the reliability, transparency, robustness, and adoption of machine learning-based medical diagnostic systems.

Accordingly, there is a need for a machine learning-based system for medical diagnosis that provides practitioners with results that are transparent and explainable. Additionally, there is a need for a system that enables practitioners to revise and supplement machine learning-based diagnoses based on their own understanding of a patient.

### SUMMARY

To overcome those and other drawbacks of prior art systems, embodiments of the disclosed system use one or more machine learning models—e.g., a bidirectional gated recurrent unit (BiGRU) model, a hybrid bidirectional long short-term memory (BiLSTM-H) model, and/or a multilabel BiLSTM (BiLSTM-M) model, a large language model (LLM)—and/or a rule-based parser to label natural language sentences (provided by patients or describing patient behaviors) as indicative of diagnostic criteria used to diagnose and/or assess the severity of a mental disorder or other medical condition.

Having identified natural language sentences as indicative of diagnostic criteria as described above, some embodiments of the disclosed system determine whether the identified diagnostic criteria are indicative of a disorder under established medical guidelines and provide a final diagnostic label. For example, consistent with the DSM5, a final diagnostic label indicative of autism spectrum disorder may be provided in response to a determination that there are examples of at least three A criteria and examples of at least two B criteria.

By outputting both the final diagnostic label and the diagnostic criteria (identified by the system and used by the system to determine the final diagnostic label), the disclosed system provides practitioners with both a diagnosis consistent with established medical guidelines and an understanding of the identified diagnostic criteria used to make that diagnosis. Accordingly, the disclosed system provides more clinical value to practitioners than the existing “black box” models used for medical diagnoses.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1 is a diagram of an architecture of the disclosed system according to exemplary embodiments.

FIG. 2A is a block diagram of the disclosed system according to an exemplary embodiment.

FIG. 2B is a block diagram of the disclosed system according to another exemplary embodiment.

FIG. 2C is a block diagram of the disclosed system according to another exemplary embodiment.

FIG. 3 includes example views of a graphical user interface according to exemplary embodiments.

FIG. 4 is a graph of illustrating the sensitivity and specificity of embodiments of the disclosed system.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

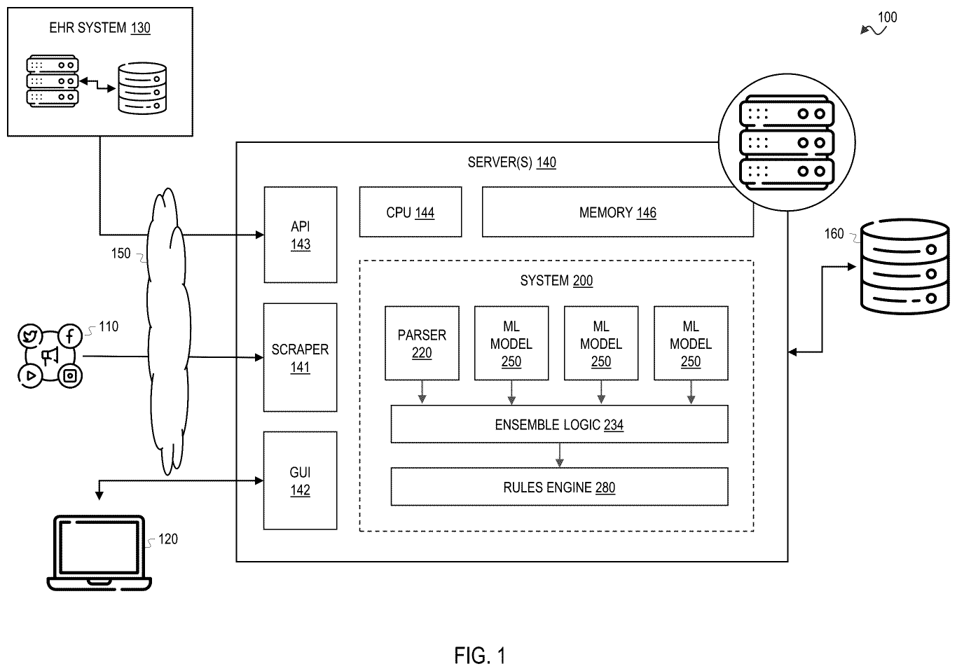

FIG. 1 is a diagram of an architecture 100 of a system 200 for automated identification of the diagnostic criteria in natural language descriptions of patient conditions or behavior according to exemplary embodiments.

As shown in FIG. 1, the architecture 100 includes at least one server 140 in communication with user devices 120 via one or more computer networks 150. The server 140 stores data in non-transitory computer readable storage media 160 and may also receive data from electronic health records systems 130. The server 140 may include an application programming interface (API) 143 that enables the server 140 to receive data from the EHR systems 130 and/or to send and receive data from the user devices 120. Additionally, the server 140 may provide a user interface 142 that enables the server 140 to provide information to and receive information from the user devices 120. Additionally, the server 140 may include a scraping module 141 that scrapes social media content (e.g., shared by caregivers) from social media networks 110.

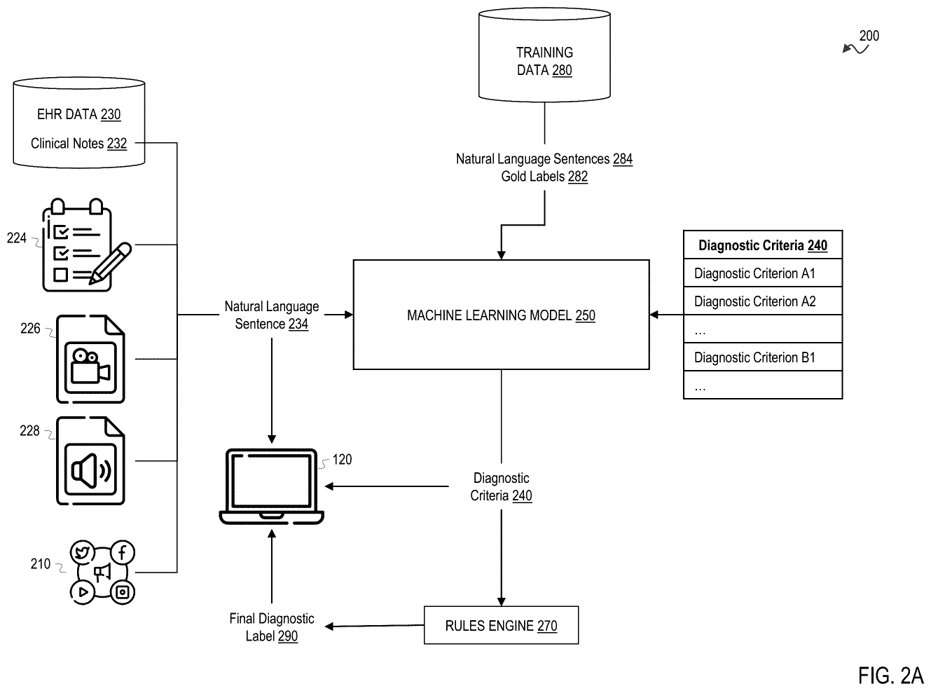

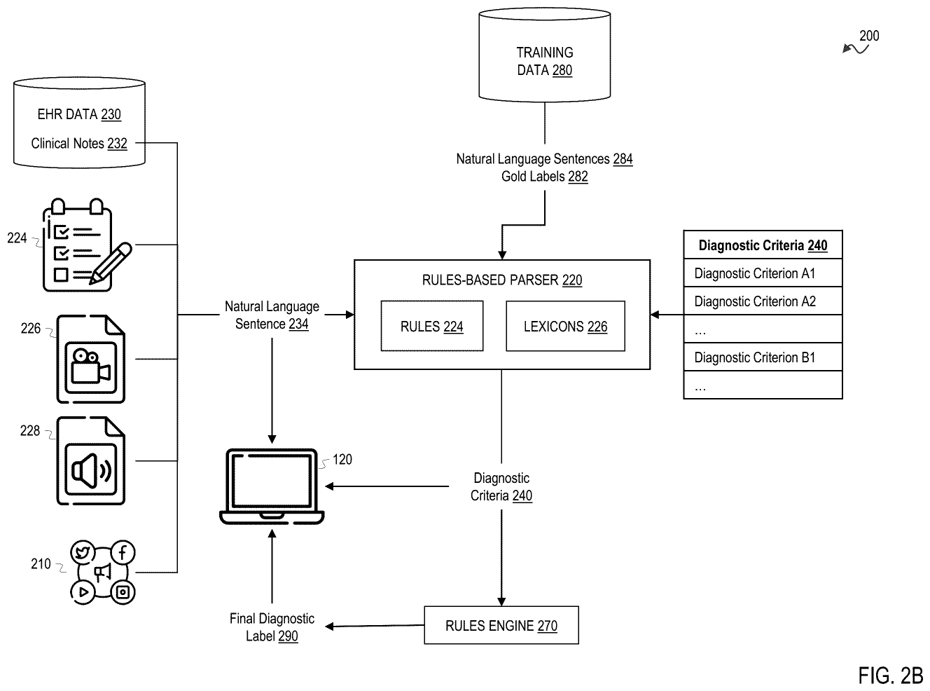

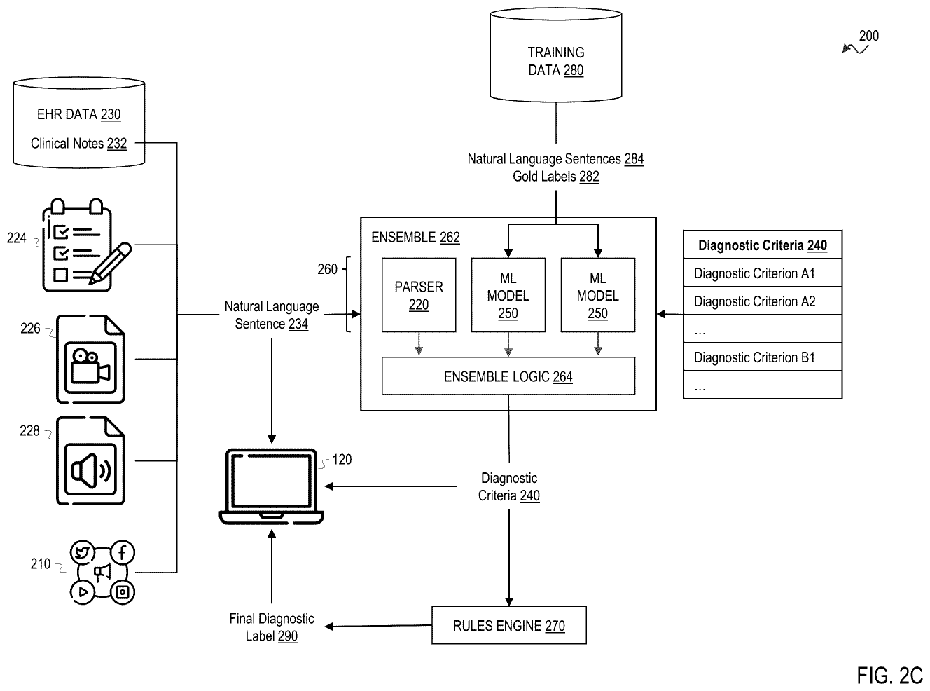

The server 140 may be any hardware computing device that stores instructions in memory 146 and includes at least one hardware computer processing unit 144 that executes those instructions to perform the functions described herein. As described in detail below with reference to FIGS. 2A-2C, embodiments of the disclosed system 200 include a rules-based parser 220, one or more machine learning models 250, a rules engine 270, etc. In some embodiments, ensemble logic 264 combines data output by the parser 220 and/or one or more machine learning models 250. Those features of the disclosed system 200 may be realized as software instructions stored (e.g., in the memory 246) and executed by the server(s) 140 (e.g., by the hardware computer processors(s) 144).

The user devices 120 may include any hardware computing device having one or more hardware computer processors that perform the functions described herein. For example, the user devices 120 may include personal computers (desktop computers, notebook computers, etc.), tablet computers, smartphones, etc. The computer network(s) 150 may include any combination of wired and/or wireless communications networks, including local area networks, wide area networks (such as the internet), etc.

FIGS. 2A-2C are block diagrams of the disclosed system 200 according to exemplary embodiments. As described in detail below, the disclosed system 200 uses one or more machine learning models 250—e.g., a bidirectional gated recurrent unit (BiGRU) model, a hybrid bidirectional long short-term memory (BILSTM-H) model, a multilabel BiLSTM (BiLSTM-M) model, a large language model (LLM), etc.—and/or a rule-based parser 220 to label natural language sentences 234 as indicative of diagnostic criteria 240 used to diagnose and/or assess the severity of a mental disorder or other medical condition.

For instance, in some embodiments, the system 200 labels individual sentences 234 as indicative of the diagnostic criteria 240 used to diagnose autism spectrum disorder as shown in Table 1:

TABLE 1

| Criterion | DSM Description (extract) | EHR Example |
|---|---|---|
| A1 | Deficits in social-emotional | He appeared fairly indifferent to his peers and did |
| | reciprocity, . . . | not initiate any interactions. |
| | | Both parent and teacher rate him as having poor |
| | | pragmatic language skills. |
| A2 | Deficits in nonverbal | Does not use appropriate social communication |
| | communicative behaviors | eye contact, etc.. |
| | used for social interaction, . . . | Does not make eye contact. |
| A3 | Deficits in developing, | During free play, he played by himself with blocks |
| | maintaining, and | and miniature replications of road signs. |
| | understanding relationships, | At home, mother reports he prefers to play by |
| | . . . | himself. |
| B1 | Stereotyped or repetitive | Mother's concerns include that she seems to repeat |
| | motor movements . . . | word a lot and will say the same sentence over and |
| | | over and over again. |
| | | She repeats phrases that are said. She is prone to |
| | | engage in repetitive activities. |
| B2 | Insistence on sameness | Both at home and school, he becomes very upset if |
| | | the routine is changed. |
| | | His mother reports that he has difficulty adapting |
| | | to changing situations and that he takes longer to |
| | | recover from difficult situations than most others |
| | | his age. |
| B3 | Highly restricted, fixated | had some difficulty interrupting this pattern once it |
| | interests that are abnormal in | started and even though she clearly enjoys joint |
| | intensity or focus | play she became fixated on her simple back and |
| | | forth movement with the objects. |
| | | She plays with the same things over and over she |
| | | is described to be fixated on superheroes |
| B4 | Hyper- or hyporeactivity to | He was observed to be sensitive to unexpected |
| | sensory input | noises by covering his ears. |
| | | He demonstrates stereotypical movements, |
| | | irregularities and impairments in communication, |
| | | and unusual responses to sensory experiences. |

In the embodiment of FIG. 2A, a machine learning model 250 may be trained using training data 280 that includes natural language sentences 284 that have been reviewed by an expert and assigned labels 282. For example, electronic health records 230 may be annotated to map individual sentences 284 in the clinical notes 232 as being indicative of one or more diagnostic criteria 240. Additionally, the electronic health records 230 may be annotated to indicate whether the identified diagnostic criteria 240 are indicative of a disorder under established medical guidelines. Additionally or alternatively, the machine learning model(s) 250 may be trained on sentences 234 provided by laypersons or patients, or deduced from direct observation, using lay language to describe examples of one or more of the diagnostic criteria 240 (or examples that are not indicative of any of the diagnostic criteria 240). Additionally, in some embodiments, the machine learning model(s) 250 may be trained using synthetic natural language sentences 234 (e.g., generated by generative AI). In each instance, the sentences 284 used to train the machine learning model(s) 250 are reviewed by an expert and provided with labels 282 as being indicative of one or more diagnostic criteria 240 (or not indicative of any of the diagnostic criteria 240).

Having been trained on the training data 280, the machine learning model 250 can be used to classify natural language sentences as being indicative of one of the diagnostic criteria 240 (or not indicative of any of the diagnostic criterion 240). For example, the machine learning model 250 can be used to characterize natural language sentences 234 extracted from the clinical notes 232 of electronic health records 230. Additionally, having been trained on natural language sentences provided by laypersons or patients using lay language, the machine learning model 250 can be used to classify natural language sentences 232 provided by caregivers and patients, for example as part of survey data 224. Additionally, the machine learning model 250 can be used to characterize natural language sentences 234 extracted from videos 226 and audio recordings 228 of patents and/or caregivers (or transcriptions of videos 226 and/or audio recordings 228) or included in social media content 210 shared by patients and/or caregivers.

Alternatively, in the embodiment of FIG. 2B, a parser 220 may use rules 224 (e.g., 193 rules) and lexicons 226 (e.g., 304 lexicons) to identify patterns in the natural language sentences 234 indicative of the diagnostic criteria 240 identified by established medical guidelines (e.g., the seven diagnostic criteria 240 for autism spectrum disorder identified in the DSM5). Each lexicon 226 may be a vocabulary of words, stemmed words, or word embeddings used to identify descriptions of a relevant concept. For instance, a lexicon 226 of terms used to identify descriptions of “excessiveness” may include “excessive”, “extremely extreme”, “above and beyond”, etc. Each rule 224 uses one or more lexicons 226 and syntactic categories that appear close together in a sentence to identify a description of a combination of such concepts (e.g., extremely sensitive to noise).

As shown in FIG. 2C, in some embodiments, multiple algorithms 260 (e.g., multiple machine learning algorithms 250, with or without the rules-based parser 220) may be used to form an ensemble 262. In those embodiments, ensemble logic 264 is used to determine whether the natural language sentence 234 is indicative of one of the diagnostic criteria 240 based on the outputs of all of the algorithms 260 in the ensemble 262. In some embodiments, for example, the ensemble logic 264 may be an ‘inclusive or’ decision where a natural language sentence 234 is assigned a DSM5 diagnostic criteria label 240 if any of the algorithms 260 assign the label 240. In other embodiments, the ensemble logic 264 may be a ‘majority vote’ decision that assigns a DSM5 diagnostic criteria label 240 to a natural language sentence 234 if a majority of the algorithms 260 assign that label 240 to the natural language sentence 234.

In various embodiments, the system 200 may be configured to label individual sentences 234 extracted from the clinical notes 232 of electronic health records 230, selected or input via the user interface 142, received via the API 143, etc. The functionality to input or select individual sentences 234 provided by the user interface 142 may include a textbox, a list of open-ended questions, a list of questions that trigger follow up questions, etc. The sentences 234 may be provided by practitioners, patients, patient caregivers, etc.

Having identified sentences 234 in the clinical notes 232 of the patient as indicative of diagnostic criteria 240 as described above, the system 200 may also include a rules engine 270 configured to determine whether the identified diagnostic criteria 240 are indicative of a disorder under established medical guidelines and provide a final diagnostic label 290. For example, consistent with the DSM5, a final diagnostic label 290 indicative of autism spectrum disorder may be provided in response to a determination that there are examples of at least three A criteria 240 and examples of at least two B criteria 240.

The system 200 outputs both the final diagnostic label 290 and the diagnostic criteria 240 (identified by the system 200 and used by the system 200 to determine the final diagnostic label 290). In various embodiments, the identified diagnostic criteria 240 and the final diagnostic label 290 may be output via a user interface 142 provided by the system 200, an email, an API 143, etc. Accordingly, the system 200 provides a transparent, understandable, and medically relevant rationale for the final diagnostic label 290 identified by the system 200. By providing practitioners with both a diagnosis 290 consistent with established medical guidelines and an understanding of the identified diagnostic criteria 240 used to make that diagnosis 290, the disclosed system 200 provides more clinical value to practitioners than the existing “black box” models used for medical diagnoses.

Additionally, outputting the diagnostic criteria 240 used to make the diagnosis 290 enables the practitioner to evaluate whether the diagnostic criteria 240 identified by the system 200 is consistent with the practitioner's assessment of the patient and, if not, whether the diagnosis 290 output by the system 200 would change under established medical guidelines if revised to conform to the practitioner's assessment. For instance, the system 200 may label a patient as having autism spectrum disorder in part because the system identifies sentences in that patient's electronic health records 230 as being indicative of B4 (i.e., “Hyper- or hyporeactivity to sensory input”). Having interacted with the patient, however, the medical practitioner may disagree that the patient exhibits that diagnostic criterion 240. Therefore, unlike with existing “black box” models, the medical practitioner can determine whether, if not for the identification of criterion B4 by the system 200, an autism spectrum disorder diagnosis is dictated under established medical guidelines.

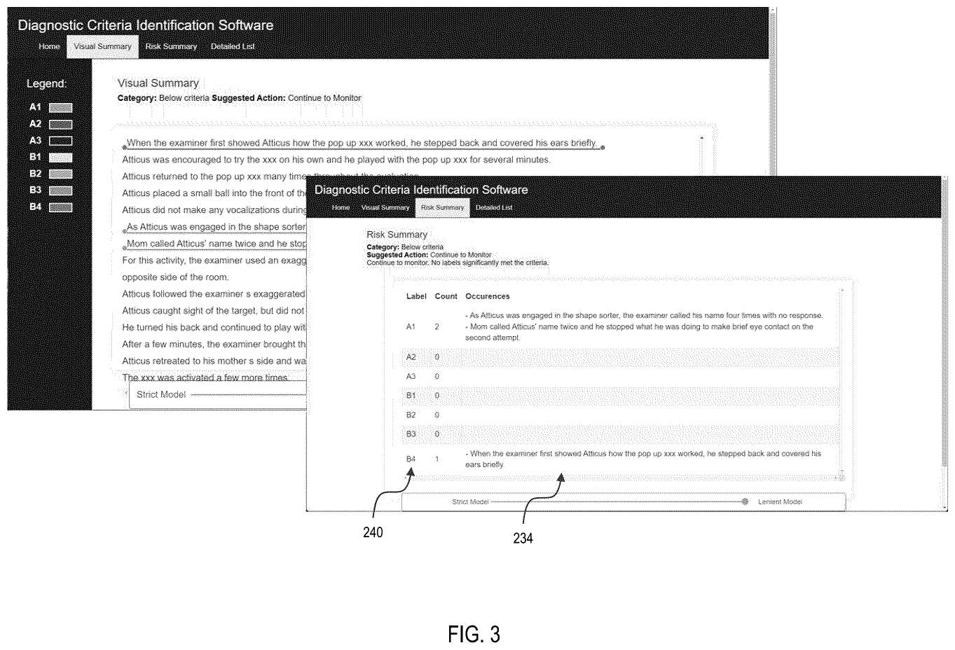

FIG. 3 is example views of a user interface 142 according to exemplary embodiments.

As shown in FIG. 3, in some embodiments, the system 200 may be configured to display the sentence 234 or sentences 234 identified by the system 200 as being indicative of each diagnostic criterion 240. Additionally, the user interface 142 may provide functionality for the medical practitioner to indicate whether, based on the practitioner's interaction with the patient, the practitioner believes the patient should be identified as exhibiting each diagnostic criterion 240. In some embodiments, the feedback provided by medical practitioners may be used to retrain the model(s) 250 to more accurately identify the diagnostic criteria. For instance, the system 200 may provide a user interface for a certified clinical reviewer to review the feedback provided by medical practitioners and determine whether to use the feedback provided by the practitioners to further refine the model(s) 250 used by the system 200.

### Evaluation of the Disclosed System

The disclosed system 200 was tested using a subset of data from the Centers for Disease Control (CDC) Autism and Developmental Disabilities Monitoring Network (ADDM), which contains information on individuals with autism, intellectual disability, and other related conditions including evaluations performed with verbatim text of results and clinical impressions as well as data for autism-specific tests performed. Starting with ADDM records with criterion labels generated by an expert using the ASD diagnostic criteria 240 in the DSM-IV, an Arizona ADDM certified clinical reviewer reviewed and annotated content for 200 records collected in 2014 and 2016 to reflect the updated to ASD diagnostic criteria 240 in the DSM-IV. Phrases 234 were mapped onto the specific DSM5 criteria 240 (A1-A3 and B1-B4) and a final diagnostic label 290 was added using DSM5 rules. Some sentences 234 received multiple criteria labels 240, which is consistent with the CDC identification research.

Of the 200 newly annotated records, 150 were used as the training data and 35 for testing as shown in Table 2. There were 18 among the 35 that received an ASD diagnosis 290 (51.4% majority baseline). The remaining 15 cases were set aside in case of a contingency or need for tuning. The records used were of children who all exhibited ASD-like behaviors, but only 51% received an ASD diagnosis 290 making this a difficult dataset for automated labeling.

TABLE 2

| Counts | Training Set (N = 150) | Test Set (N = 35) |
|---|---|---|
| Sentences | 34,313 | 6,773 |
| A1 labels | 855 | 224 |
| A2 labels | 471 | 109 |
| A3 labels | 524 | 143 |
| B1 labels | 539 | 113 |
| B2 labels | 338 | 105 |
| B3 labels | 146 | 69 |
| B4 labels | 697 | 151 |

A rule-based parser 220 and three deep machine learning networks 250 were selected after a variety of versions and settings were evaluated. These individual components 260 were also consolidated into two types of ensembles 232 as described below.

The rule-based parser 220, developed in Java, was originally created for the autism criteria in the DSM-IV and was adjusted for the DSM5 criteria 240. New rules 224 were created in two phases each analyzing twenty annotated electronic health records 230 at a time. In the first phase, rules 224 and lexicons 226 were adjusted to develop patterns for the seven DSM5 criteria 240. Then, rules 224 and lexicons 226 were further optimized using an additional 20 annotated electronic health records 230. The evaluation was done on new records 230. The rules-based parser 220 leveraged 193 rules 224 and 304 lexicons 226. The patterns identify different behaviors matching DSM criteria 240 A1-A3 and B1-B4.

A BiGRU (Bidirectional Gated Recurrent Unit) was developed and trained using TensorFlow. This version was trained separately for each criterion label (A1-A3, B1-B4). In pilot work, we evaluated training this model 250 for multi-labeling, i.e., the algorithm is trained to assign all labels, but found that the performance was lower than training per label. The model 250 used was trained for two epochs (128 GRUs, 9 layers, batch size=1000) and uses Global Vectors (GloVe) pretrained embeddings (300 dimensions), the embeddings contain 6 billion tokens and a 400 k vocabulary.

A hybrid Bidirectional Long Short-term Memory (BILSTM-H) neural network 250 was trained using TensorFlow to identify criterion labels 240 (A1-A3, B1-B4). Expert knowledge was incorporated in the BILSTM model by adding information from the rule-based parser 220. Specifically, terms matching the parser 220 lexicons 226 were concatenated to the input and parser tags. A small increase in performance was found with this additional information over a traditional BILSTM and, therefore, it was used in the ensembles 232. The best model 250 was trained for 10 epochs (batch size=16, Bidirectional units=150, parser input units=10, attention layer units=20, final layer units=150)) and using GloVe embeddings (300 dimensions).

A multilabel BiLSTM (BiLSTM-M) neural network 250 was trained using Tensorflow without parser input. After testing different thresholds, the BILSTM version that performed best assigned DSM5 criterion labels 240 when the predicted value (a threshold in the model 250) was set at 0.5. Other parameters were the same as for the BILSTM-H above.

Finally, the individual algorithms 260 were combined in ensembles 232, a common approach to combining input from different algorithms 260. Six ensembles 232 were compared. They differ in the algorithms 260 included and the ensemble logic 264 used to combine the output of the individual algorithms 260. A first ensemble 262 combined all algorithms 260 developed, a second ensemble 262 combined only the ML models 250 (i.e., no parser 220), and a third ensemble 262 combined the two top performing ML models 250. Each ensemble 262 used two approaches to combine output. The first ensemble logic 264 tested was an ‘inclusive or’ decision where a sentence 234 was assigned a DSM5 diagnostic criteria label 240 if any of the algorithms 260 assign the label 240. That was the least restrictive model and would assign a label 240 to a sentence 234 if any of the algorithms 260 assigned that label 240. The second ensemble logic 264 evaluated was a ‘majority vote’ decision, which assigned a DSM5 diagnostic criteria label 240 to a sentence 234 if the majority of the algorithms 260 assigned that label 240 to the sentence 234. That was a strict model wherein multiple individual components 260 needed to agree on a label 240.

### Results

Five common measures of model performance were calculated, defined using true positive (TP), true negative (TN), false positive (FP), and false negative (FN), actual positive (P), actual negative (N), predicted positive (PP), and predicted negative (PN) counts:

$$\begin{matrix}\cdot & Accuracy = ( TP + TN ) / ( P + N ) \\ \cdot & Precison or Positive Predictive Value ( PPV ) = TP / PP \\ \cdot & Sensitivity or Recall = TP / P \\ \cdot & F 1 = 2 ⋆ TP / ( 2 ⋆ TP + FP + FN ) \\ \cdot & Specificity = TN / N\end{matrix}$$

Precision (or positive predictive value) and recall (or sensitivity) are commonly used in both ML and medicine. F1 is the harmonic mean of precision and recall and indicates how well-balanced a system is. The F1 value will be low if either precision or recall are low. These are the most informative for our sentence labeling.

Accuracy is a typical ML metric indicating how well a decision is made out of all decisions that the ML made. This measure is especially important for case labeling since it shows how much better an algorithm performs against the majority baseline, i.e., assigning the most common label (51% in our test set since there are 18 ASD cases among 35). Finally, specificity is included as a typical metric in medicine.

The best performing ML model was also compared with autism-specific diagnostic test results, available in ADDM data, using chi-squared analysis.

The performance of the four algorithms 260 when assigning individual criteria labels 240 to a sentence 234 is briefly described below.

When averaging over all 7 different diagnostic criteria 240, average precision was the highest for the multilabel LSTM (67%), average recall was the highest for the hybrid LSTM (52%), and the multilabel LSTM achieved the highest F-measure (0.57). The parser 220 scored higher on precision (49%) than on recall (35%) for individual criteria 240 and scored lower than the ML models 250. The BiGRU showed 58% precision and 47% recall, the hybrid LSTM was more balanced with 54% precision and 52% recall, and the multilabel LSTM scored 67% precision and 51% recall.

The BiGRU was unable to label any sentences for the B3 criteria 240, resulting in a score of 0 for each measure for B3 (and so lowering average precision and recall). Most models 250 performed worse for that criterion 240, and it is the criterion 240 for which the fewest examples were available in the training data 280. All models 250 performed well for the A2 criteria 240, even though this is not the criteria 240 with the most examples in the training data 280.

Table 3 shows the results for the ensembles 232 for the individual diagnostic criteria 240 and the averages. Results for combining algorithms 260 using an inclusive-or ensemble logic 264 showed the highest F-measure (0.58) was achieved when the top ML models 250 were combined. That combination also led to the highest precision (61%). However, the highest recall (70%) was found when the four algorithms 260 were combined. The same combinations were tested with a majority-vote ensemble logic 264. The highest F-measure (0.58) and the highest recall (51%) were found when the ML algorithms 250 were combined. However, the highest precision (76%) was found when the parser 220 and ML algorithms 250 were combined.

TABLE 3

| All Four Algorithms | | | All ML Algorithms | | | Top Two Algorithms | | |
|---|---|---|---|---|---|---|---|---|
| P | R | F1 | P | R | F1 | P | R | F1 |

Exclusive Or

| | | | | | | | | | |
|---|---|---|---|---|---|---|---|---|---|
| A1 | 0.38 | 0.74 | 0.50 | 0.42 | 0.71 | 0.53 | 0.54 | 0.61 | 0.57 |
| A2 | 0.52 | 0.78 | 0.62 | 0.58 | 0.73 | 0.65 | 0.69 | 0.50 | 0.58 |
| A3 | 0.41 | 0.61 | 0.49 | 0.55 | 0.57 | 0.56 | 0.64 | 0.52 | 0.58 |
| B1 | 0.42 | 0.79 | 0.55 | 0.50 | 0.73 | 0.59 | 0.61 | 0.68 | 0.64 |
| B2 | 0.46 | 0.64 | 0.54 | 0.55 | 0.60 | 0.58 | 0.71 | 0.53 | 0.61 |
| B3 | 0.46 | 0.57 | 0.51 | 0.48 | 0.52 | 0.50 | 0.59 | 0.39 | 0.47 |
| B4 | 0.40 | 0.81 | 0.53 | 0.44 | 0.79 | 0.57 | 0.47 | 0.77 | 0.58 |
| Avg. | 0.44 | 0.70 | 0.54 | 0.50 | 0.67 | 0.57 | 0.61 | 0.57 | 0.58 |

Majority-vote ensembles

| | | | | | | | | | |
|---|---|---|---|---|---|---|---|---|---|
| A1 | 0.76 | 0.40 | 0.53 | 0.63 | 0.54 | 0.58 | 0.78 | 0.39 | 0.52 |
| A2 | 0.98 | 0.54 | 0.70 | 0.78 | 0.60 | 0.68 | 0.95 | 0.50 | 0.66 |
| A3 | 0.81 | 0.36 | 0.50 | 0.75 | 0.45 | 0.57 | 0.80 | 0.38 | 0.52 |
| B1 | 0.73 | 0.52 | 0.61 | 0.71 | 0.58 | 0.64 | 0.82 | 0.52 | 0.64 |
| B2 | 0.82 | 0.39 | 0.53 | 0.79 | 0.46 | 0.58 | 0.82 | 0.40 | 0.54 |
| B3 | 0.56 | 0.07 | 0.13 | 0.66 | 0.28 | 0.39 | 0.00 | 0.00 | 0.00 |
| B4 | 0.67 | 0.56 | 0.61 | 0.60 | 0.69 | 0.64 | 0.64 | 0.60 | 0.62 |
| Avg. | 0.76 | 0.41 | 0.51 | 0.70 | 0.51 | 0.58 | 0.69 | 0.40 | 0.50 |

When comparing the two types of ensemble logic 264, the ensembles 232 using majority vote logic 264 showed higher precision (76% for the four algorithms 260, 70% for the ML algorithms 250, and 69% the top two algorithms 260). The ensembles 232 using inclusive-or logic 264 showed higher recall (70% for the four algorithms 260, 67%, for the ML algorithms 250, and 57% for the top two algorithms 260).

Based on the labels for each individual diagnostic criterion 240, each test case was assigned a final diagnostic label 290 for ASD or no ASD. The outcomes for the four individual algorithms 260 and for the different ensembles 232 were compared. Overall, the best performance was achieved with the ensemble 262 containing the two top ML algorithms 250 (BiGRU and multilabel LSTM), which achieved 100% precision, 83% recall (or sensitivity), and 100% specificity. Its accuracy of 91% was much higher than the majority baselines of 51.4% (18 out of 35 cases labeled ASD). Among the single algorithms 260, the BiGRU by itself achieved the highest performance, with 89% for each measure except 88% for specificity.

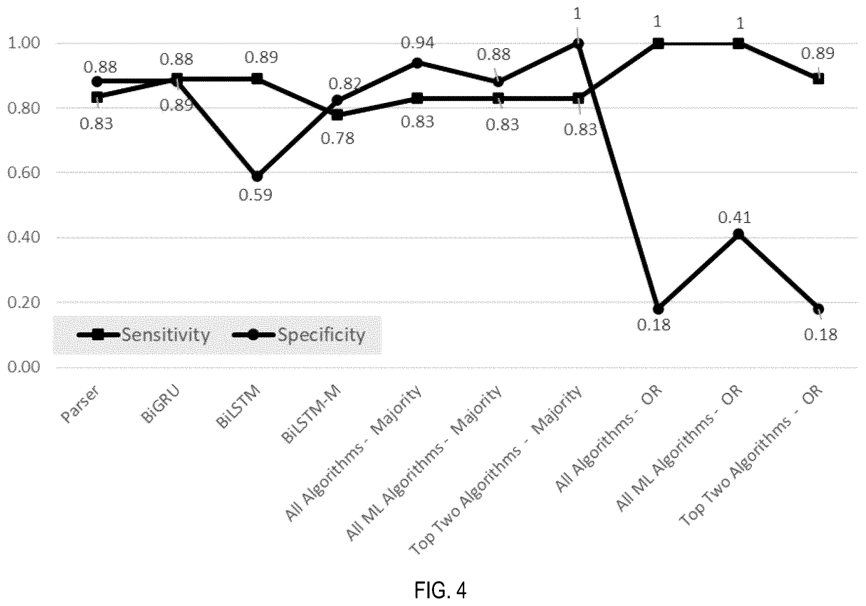

FIG. 4 is a graph illustrating the sensitivity and specificity of the disclosed parser 220 and machine learning models 250 individually and as ensembles 232 having either of the two ensemble logics 234. As shown in the FIG. 4, while most algorithms 260 performed well on sensitivity, combining algorithms into an ensemble 262 while using the majority vote ensemble logic 264 improved specificity.

The original dataset also included the results for autism-specific diagnostic instruments when they were administered. The results of the disclosed system 200 were compared with the decision that could be made using the diagnostic instruments. Of the 35 cases in the test set, there were 30 where at least one diagnostic instrument was applied and 14 wherein the actual results were stated in the record. The results of those tests were used to assign a label to the cases as follows:
Asperger Syndrome Diagnostic Scale (ASDS): Scores >110 were labeled as ASD, scores between 90-110 inclusive as were labeled Probable ASD, all else were labeled as Not ASD,
Childhood Autism Rating Scale (CARS): All results listed as ASD (Severe, Mild to Moderate, NOS) were labeled as ASD, all else were labeled as Not ASD,
Gilliam Asperger Disorder Scale (GADS): Scores High/Probable were labeled as ASD, scores listed as Borderline were labeled as Probable ASD, all else were labeled as Not ASD,
Gilliam Autism Rating Scale (GARS): Scores >110 were labeled as ASD, scores between 80-110 inclusive were labeled as Probable ASD, all else were labeled as Not ASD,
GARS2: Scores >=85 were labeled as ASD, all else were labeled as Not ASD,
Modified Checklist for Autism in Toddlers (MCHAT): Scores >=2 were labeled as ASD, all else were labeled as Not ASD,
Autism Diagnostic Observation Schedule (ADOS) was scored the same as the ML models—ASD or Not ASD,

The results of existing diagnostic tests were compared to the disclosed ML ensemble 262. When any diagnostic test indicated autism, sensitivity was low and specificity high. The ensemble 262 that used majority vote logic 264 performed with higher sensitivity (0.83) and specificity (1.0) than the best diagnostic tests. Table 4 provides chi-square test results for independence results for the ASD diagnostic tests and the best performing ensemble 262 of the disclosed system 200 for the expected counts of ASD and No ASD. There was no significance for the Diagnostic Test group whereas the best performing ensemble 262 of the disclosed system 200 was much more likely to rule out ASD. (P=0.01, standardized residuals 2.32).

TABLE 4

| | | Gold Standard Label | | |
|---|---|---|---|---|
| | | ASD | No ASD | P- |
| | | N = 11 | N = 3 | value |
| Diagnostic Tests | ASD | 7 (63.6%) | 2 (66.7%) | 1.00 |
| | No ASD | 4 (36.4%) | 1 (33.3%) | |
| Majority Vote Ensemble | ASD | 10 (90.9%) | 0 (0.00%) | 0.011 |
| of Top 2 Algorithms | No ASD | 1 (9.09%) | 3 (100%) | |

While the disclosed system 200 is described above in reference to diagnosing autism spectrum disorder, one of ordinary skill in the art would recognize that the disclosed system 200 can be configured to identify sentences 234 (e.g., in clinical notes 232) indicative of other diagnostic criterion 240 used to diagnose other mental disorders. While preferred embodiments have been described above, those skilled in the art who have reviewed the present disclosure will readily appreciate that other embodiments can be realized within the scope of the invention. Accordingly, the present invention should be construed as limited only by any appended claims.

What is claimed is:

1. A method, comprising:
parsing individual sentences, by a machine learning model trained using annotated natural language examples, to identify sentences indicative of diagnostic criteria used to diagnose a mental disorder or medical condition under established medical guidelines;
identifying a final diagnostic label for a patient by determining whether, under the established medical guidelines, the identified diagnostic criteria are indicative of the mental disorder; and
outputting the final diagnostic label and each of the identified diagnostic criteria.

2. The method of claim 1, wherein some of the natural language examples are:
extracted from electronic health records of patients diagnosed with the medical condition;
sentences used by patients diagnosed with the medical condition; or
sentences used to describe behavior or symptoms of patients diagnosed with the medical condition.

3. The method of claim 2, wherein the annotated natural language examples are labeled by an expert as being indicative of one or more of the diagnostic criteria or not indicative of any diagnostic criterion.

4. The method of claim 1, wherein the individual sentences are extracted from electronic health records of the patient.

5. The method of claim 1, wherein the individual sentences are received via a user interface, an application programming interface, extracted from social media content shared by patient caregivers or patients, or extracted from videos or audio recordings of patents or patient caregivers.

6. The method of claim 1, further comprising:
outputting the sentences identified as indicative of each of the identified diagnostic criteria.

7. The method of claim 6, further comprising:
providing functionality for a medical practitioner to modify each of the identified diagnostic criteria.

8. The method of claim 7, further comprising:
identifying a revised diagnostic label for the patient by determining whether, under the established medical guidelines, the modified diagnostic criteria are indicative of the medical condition.

9. The method of claim 8, further comprising:
labeling the sentences identified by the machine learning model using the modified diagnostic criteria; and
training the machine learning model using the sentences identified by the machine learning model and labeled using the modified diagnostic criteria.

10. The method of claim 1, wherein the machine learning model comprises a bidirectional gated recurrent unit (BiGRU) model, a hybrid bidirectional long short-term memory (BiLSTM-H) model, a multilabel BILSTM (BiLSTM-M) model, or a large language model (LLM).

11. A system, comprising:
non-transitory computer readable storage media; and
at least one hardware computer processor configured to:
parse individual sentences, by a machine learning model trained using annotated natural language examples, to identify sentences indicative of diagnostic criteria used to diagnose a mental disorder or medical condition under established medical guidelines;
identify a final diagnostic label for a patient by determining whether, under the established medical guidelines, the identified diagnostic criteria are indicative of the mental disorder; and
output the final diagnostic label and each of the identified diagnostic criteria.

12. The system of claim 11, wherein some of the natural language examples are:
extracted from electronic health records of patients diagnosed with the medical condition;
sentences used by patients diagnosed with the medical condition; or
sentences used to describe behavior or symptoms of patients diagnosed with the medical condition.

13. The system of claim 12, wherein the annotated natural language examples are labeled by an expert as being indicative of one or more of the diagnostic criteria or not indicative of any diagnostic criterion.

14. The system of claim 11, wherein the individual sentences are extracted from electronic health records of the patient.

15. The system of claim 11, wherein the individual sentences are received via a user interface or an application programming interface, extracted from social media content shared by patient caregivers or patients, or extracted from videos or audio recordings of patents or patient caregivers.

16. The system of claim 11, wherein the at least one hardware computer processor is further configured to:
output the sentences identified as indicative of each of the identified diagnostic criteria.

17. The system of claim 16, wherein the at least one hardware computer processor is further configured to:
provide functionality for a medical practitioner to modify each of the identified diagnostic criteria.

18. The system of claim 17, wherein the at least one hardware computer processor is further configured to:
identify a revised diagnostic label for the patient by determining whether, under the established medical guidelines, the modified diagnostic criteria are indicative of the medical condition.

19. The system of claim 18, wherein the at least one hardware computer processor is further configured to:
label the sentences identified by the machine learning model using the modified diagnostic criteria; and
train the machine learning model using the sentences identified by the machine learning model and labeled using the modified diagnostic criteria.

20. The system of claim 11, wherein the machine learning model comprises a bidirectional gated recurrent unit (BiGRU) model, a hybrid bidirectional long short-term memory (BiLSTM-H) model, a multilabel BiLSTM (BiLSTM-M) model, or a large language model (LLM).
